(19) United States
(12) Patent Application Publication
(10) Pub. No.: US 2003/0108997 A1
(43) Pub. Date: Jun. 12, 2003

(54) Optimizing temperature shifting in high cell density fermentations

(76) Inventors: Klaus Di. Mahr, Hochheim (DE); Guenter Eisbrenner, Frankfurt (DE); Herbert Maerkl, Seevetal (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/245,048

(22) Filed: Sep. 17, 2002

Related U.S. Application Data
(60) Provisional application No. 60/346,113, filed on Jan. 4, 2002.

(30) Foreign Application Priority Data
Sep. 20, 2001 (DE) 10146445.2-41
Sep. 6, 2002 (WO) PCT/EP02/09976

(51) Int. Cl. C12P 21/02; C12N 1/21

(52) U.S. Cl. 435/69.1; 435/252.33

(57) ABSTRACT

The invention relates to a fermentation method for improving the expression of a heterologous protein in E. coli cells which requires a slow temperature shift downward during the expression phase, beginning at 35° C. with the temperature being lowered in small increments (from 0.2 to 0.8° C.) at 10 to 30 minutes intervals, until the temperature reaches 26° C. The temperature shift begins 20 to 60 minutes following induction of the expression phase.

### BACKGROUND OF THE INVENTION

The temperature constitutes an important parameter in the bioprocess technology. It affects the rate of growth and metabolic processes of microorganisms. In many production methods using microorganisms different temperature ranges are used during the course of the process with it being possible to shift the temperature both upwards and downwards. Thus, in methods in which secondary metabolites are produced, two temperature ranges are frequently used. Temperatures which are optimal for growth are used in the growth phase during which the mycelium is formed. This achieves maximal specific growth rates. In the production phase, the given temperature is lowered from approximately 2° C. to 5° C. downward to a temperature, which is optimal for producing the secondary metabolite (e.g. antibiotic). The specific growth rates obtained are submaximal. Mycelial growth reaches a static state in which the rate of formation and the rate of decay are in equilibrium. Using these fermentation conditions, it is possible to achieve high yields.

Another example in which temperature shifting is employed is in the preparation of interferon (European patent No. 0058687) using diploid human cells. In this case, the temperature is shifted both upwards and downwards. In phase 1, the cells are induced at 34° C. In phase 2, the temperature is shifted up to 37° C. for about 2 hours to initiate the production of interferon. In phase 3 the temperature is shifted down to the interferon production temperature of 30° C. The major part of the interferon yield is then produced at this temperature.

Temperature shifts are frequently used in fermentations for many different purposes such as preparing enzymes, increasing the production of biomass, reducing the quantity of unwanted by-products or as methods of induction. Consequently, it is the state of the art to use temperature shifts in the bioprocess technology with the temperatures being shifted very rapidly from one range to another. This can in practice be regarded as being a step function of the temperature.

Shifting the temperature in accordance with the above-mentioned insights was ineffective in fed-batch fermentations using recombinant E. coli where inducers such as isopropy-beta-thio-galactoside (IPTG) or indolic-propionic acid (IPA) are used to initiate expression. The reason for this is that the induction rather than the temperature affect the submaximal specific growth rates of the strains. Accordingly, there is a need in the art to improve the fermentation conditions for the expression and production of heterologous protein in microorganisms, in particular, E. coli cells.

### SUMMARY OF THE INVENTION

A novel fermentation method has been identified that that improves the expression of a heterologous protein in E. coli cells. The method comprising the steps of growing E. coli cells in culture in the growth phase until cell mass is predominantly at maximum specific growth rate, and slowly shifting the temperature downward in the expression phase from 35° C. down to 26° C. starting from 20 to 60 minutes after induction in 0.2° C. to 0.8° C. temperature increments at 10 to 30 minute intervals such that said expression of heterologous protein is increased. Other aspects of the inventions are described hereinbelow.

### BRIEF DESCRIPTION OF THE FIGURES

FIG. 1 is a schematic drawing of a membrane bioreactor.

FIG. 2 depicts the fermentation at a constant temperature of 35° C.

FIG. 3 depicts the fermentation with rapid lowering of the temperature from 36° C. to 26° C. in accordance with the prior art.

FIG. 4 depicts the fermentation with rapid lowering of the temperature from 36° C. to 26° C. in accordance with the instant invention.

### DETAILED DESCRIPTION

Fermentation methods are divided into two phases. Phase 1 is the growth phase in which cell mass is predominantly formed at the maximum specific growth rate. Phase 2 takes place following induction and is the transition from a cell growth to the expression or production of heterologous protein. When inducer is added and the temperature level is unchanged, respiration, metabolism and cell growth immediately decelerates. In this phase cells are producing recombinant protein at a submaximal specific growth rate. If in this state the temperature is additionally shifted very rapidly downwards, the entire process comes to a standstill.

Shifting the temperature rapidly from 35° C. to 26° C. was also ineffective in improving the expression of heterologous protein in the case of high cell density fermentations prepared in 2 L membrane dialysis fermenters (compare Examples 1 and 2 in the example section).

Applicants have considered the question of how the temperature shift might be configured differently, for example, by carrying out the temperature shift such that negative influences do not arise either in connection with dry biomass (DBM) production or with fusion protein (FP) production. Since there is a slight steady decline in the growth rate during the induction phase when the process is carried out at constant temperature, Applicants attempted to optimize growth and production conditions by cooling steadily in a manner which is matched to the growth rate of the organisms. To do this, the temperature was decreased, from 35° C. down to a final temperature of 26° at a rate of 1.5° C. per hour beginning forty minutes after induction. In the experimental fermentation described in Example 4, an optimized temperature profile was found in which the contents of DBM and FP was surprisingly very high. 125.9 g of DBM/l and 27.9 g of FP/l were produced using the fermentation conditions described in Example 4. Furthermore, the specific productivity was 222 mg of FP/g of DBM.

Consequently, the present invention relates to a fermentation method for expressing a heterologous protein in E. coli cells while slowly shifting the temperature during the expression phase, with the temperature being lowered, in small increments of from 0.2° C. to 0.8° C., preferably 0.5° C., at intervals of from 10 to 30 minutes preferably at 20 minutes, intervals, from 35° C. down to 26° C. starting at 20 to 60 minutes, preferably 40 minutes after induction. The E. coli cells are preferably cultured in membrane dialysis fermenters having a capacity of more than 2 L, preferably comprising the dialysis membrane being installed internally or externally on the fermenter; preferably comprising the membranes having a cut-off of at least 10 kDal, in particular comprising the induction being carried out at an optical density of from 90 to 110, preferably at 100, more preferably comprising, after the temperature has been shifted down to 26° C., this temperature being maintained until the end of the culture; particularly comprising using a minimal medium; in particular comprising the carbohydrate concentration being from 0.1 to 1.0 g/l; and especially comprising the oxygen saturation being >10%.

### EXAMPLES

When carrying out the experimental fermentations in the examples below, the preparations of the fermenter unit, the respective inoculum, the fermentation medium and the trace element solution were identical.

### Experimental Conditions for Examples 1-3

Preliminary Culture and Inoculum

The inoculation of the preliminary culture took place, under a laminar air flow hood, in a 500 ml shaker flask, which was fitted with baffles and filled with 100 ml of the sterilized preliminary culture medium, using a working seed (1 ml) which had been cryostated at −80° C.

Composition Based on 1 L of Medium:

| | |
|---|---|
| NaCl | 3.50 g |
| Yeast extract | 8.00 g |
| Glucose | 1.00 g |
| K₂HPO₄ | 3.68 g |
| KH₂PO₄ | 1.32 g |
| In H₂O | |

The preliminary culture was incubated for 3.25 h, at 37° C. and 200 rpm, on an orbital shaker. After a 1 ml sample had been removed, the optical density at 540 nm was determined using a 1:10 dilution. The measured optical density is used to compute the inoculation volume in each case.

Fermentation Medium

| | | |
|---|---|---|
| Glucose monohydrate | 38.62 | g/l |
| Citric acid × H₂O | 3.53 | g/l |
| NaOH (33% strength) | 1.15 | g/l |
| H₃PO₄ (85% strength) | 2.00 | ml/l |
| K₂HPO₄ | 1.18 | g/l |
| MgSO₄ × 7H₂O | 2.00 | g/l |
| Na₂SO₄ | 3.00 | g/l |
| Trace element solution | 10.00 | ml/l |
| Fe₂(SO₄)₃ × H₂O | 0.50 | g/l |
| Desmophene | 0.25 | ml/l |
| in H₂O_{distilled} | | |

Prior to sterilization, the pH was adjusted to 3.5 using 33% NaOH. After the sterilization, the pH was adjusted to 7.1 using 25% NH₃ solution. After sterilization, 5 ml of thiamine HCl (5 g/l) were added to the medium in the inner space.

Trace Element Solution (100-Fold Concentration):

| | |
|---|---|
| H₃PO₄ | 2.000 g/l |
| CuSO₄ × 5H₂O | 0.160 g/l |
| MnSO₄ × H₂O | 1.232 g/l |
| ZnSO₄ × 7H₂O | 1.600 g/l |
| (NH₄)₆Mo₇O₂₄ × 4H₂O | 0.800 g/l |
| KI | 0.400 g/l |

Preparing the Fermenter Unit

A membrane bioreactor (Bioengineering, Wald, Switzerland) having an outer film of 80 μm in thickness (Naturin) was used for the fermentation.

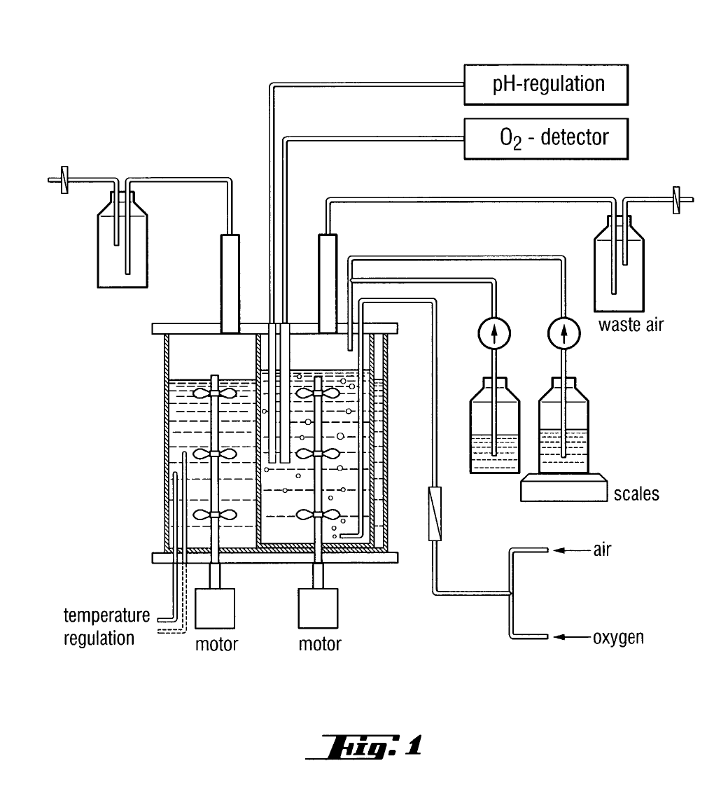

A cuprophane dialysis membrane (Akzo), having a wall thickness of 20 μm and a molecular cut-off of 10 kDal, was used for separating the fermentation space and the dialysate space (FIG. 1).

The unit was sterilized in situ together with the fermentation medium at 121° C. for 20 min. Prior to the sterilization, the volume in the inner space was 1,100 ml while that in the outer space was 3,900 ml. The connections for gassing, for exhaust air, for withdrawing samples from the inner space and the outer space, and for adding concentrated feed and ammonia solution, were sterilized separately in the autoclave at 121° C. for 20 min and after the fermenter had been sterilized attached using diaphragms while flaming. After sterilization, medium was drained from the inner space in order to compensate for medium which had passed from the outer space to the inner space during the sterilization and to adjust the volume once again to 1,100 ml. The pH was adjusted to 7.1 using 25% NH₃ solution. 5 ml of thiamine HCl solution (5 g/l) was added to the inner space.

### Example 1

### Experiment at Constant Fermentation Temperature of 35° C.

The sterilized medium was inoculated with 9 ml of inoculum. The fermentation was operated batchwise until the glucose concentration in the inner space in the fermenter had fallen below a value of 3 g/l. After the experiment had been in progress for 17.31 hours, concentrated glucose solution into the inner space was monitored in the fermenter using a Watson Marlow peristaltic pump. The concentration of glucose in the inner space in the fermenter was subsequently determined regularly and the substrate concentration was kept in the region of 0.5 g of glucose/l by manually adjusting the rotational speed of the pump.

After 18.3 hours, induction took place, at an optical density of OD₅₄₀=95.5, by adding 3 ml of 30% IPTG solution, which had been sterilized by filtration, to the inner space in the fermenter and adding 10 ml of 30% IPTG solution to the outer space in the fermenter.

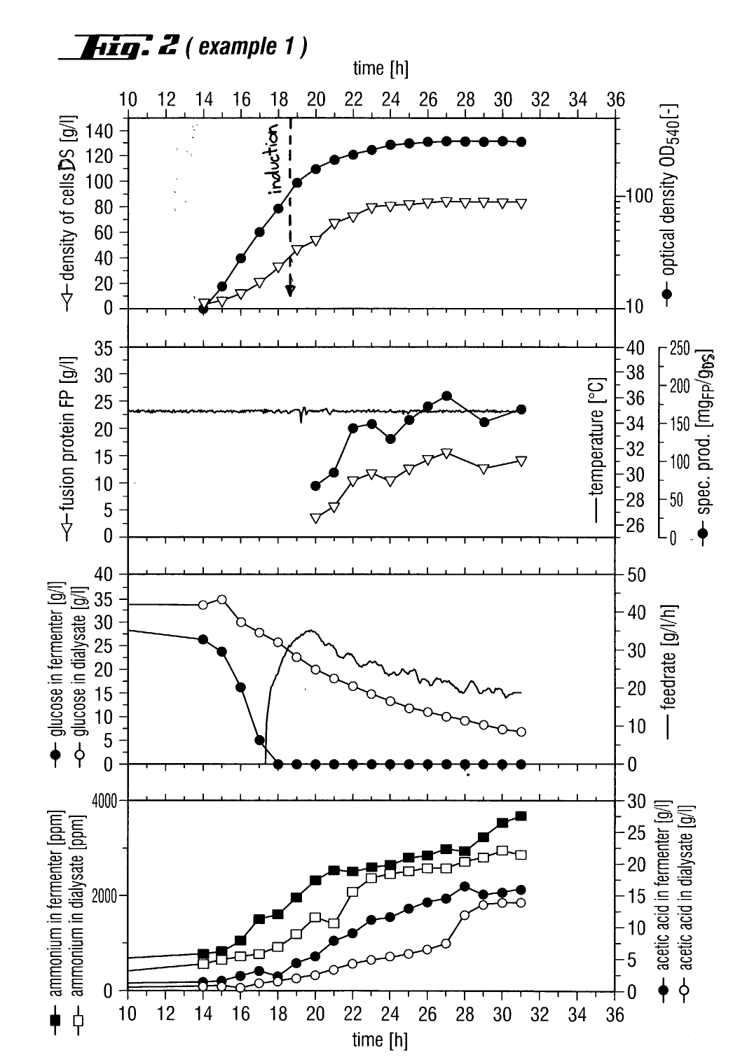

Culturing took place at a stirrer speed of 1,500 rpm in the inner space in the fermenter and 800 rpm in the outer space in the fermenter, at 35° C. and an oxygen saturation of >10%. The pH was measured using a combination electrode supplied by Broadley James, USA. The pH was adjusted online to 7.1 with the aid of a Bioengineering pH controller and by metering in 25% NH₃ solution using a Meredes peristaltic pump. The fermentation ended after 31 hours. During the 27th hour, the dry matter amounted to 84 g/l (maximum) and the product concentration was 15.8 g of fusion protein/l. The chronological course of the fermentation is depicted in FIG. 2. (DS in FIGS. 2-4 legends stand for dry substance.)

At the time of induction, the maximum specific growth rate was μₘₐₓ=0.511/h, while it declined to μ=0.282/h after 2 hours of expression and subsequently, up to the 27th hour, at which productivity was maximal, declined steadily down to μ=0.013/h. The maximum formation of acetate amounted to 16.5 g/l

### Example 2

### Rapid Lowering of the Temperature, in Accordance with the Prior Art, from 36° C. Down to 26° C.

The inoculation volume of 12 ml was transferred to the fermenter using a sterile tip while flaming. The fermentation was operated batchwise until the glucose concentration in the inner space in the fermenter had fallen below a value of 3 g/l. After the experiment had been in progress for 15.28 hours, concentrated glucose solution was added into the inner space in the fermenter using a Watson Marlow peristaltic pump. The concentration of glucose in the inner space in the fermenter was subsequently determined irregularly and the substrate concentration was kept in the range of from 0.5 to 1.0 g of glucose/l by manually adjusting the pump speed.

After 15.85 hours, the induction was effected, at an optical density of OD₅₄₀=92, by adding 3.5 ml of 30% IPTG solution, which had been sterilized by filtration, to the inner space in the fermenter and adding 10.5 ml of 30% IPTG solution to the outer space in the fermenter. The stirrer speed was 1,500 rpm in the inner space in the fermenter and 800 rpm in the outer space in the fermenter. During the course of the fermentation, the stirrer speed in the inner space in the fermenter was increased to a maximum of 2,300 rpm.

Up to 30 min after the induction (16.35 hours), the temperature was 35° C. The required temperature value was then adjusted to the final value of 26° C. and kept constant until the end of the fermentation. The down-shifted temperature of 26° C. was reached after 0.48 hours. The pH was measured using a Broadley combination electrode. The pH was adjusted online to 7.1 with the aid of a Bioengineering pH controller and by metering in 25% NH₃ solution using a Meredes peristaltic pump.

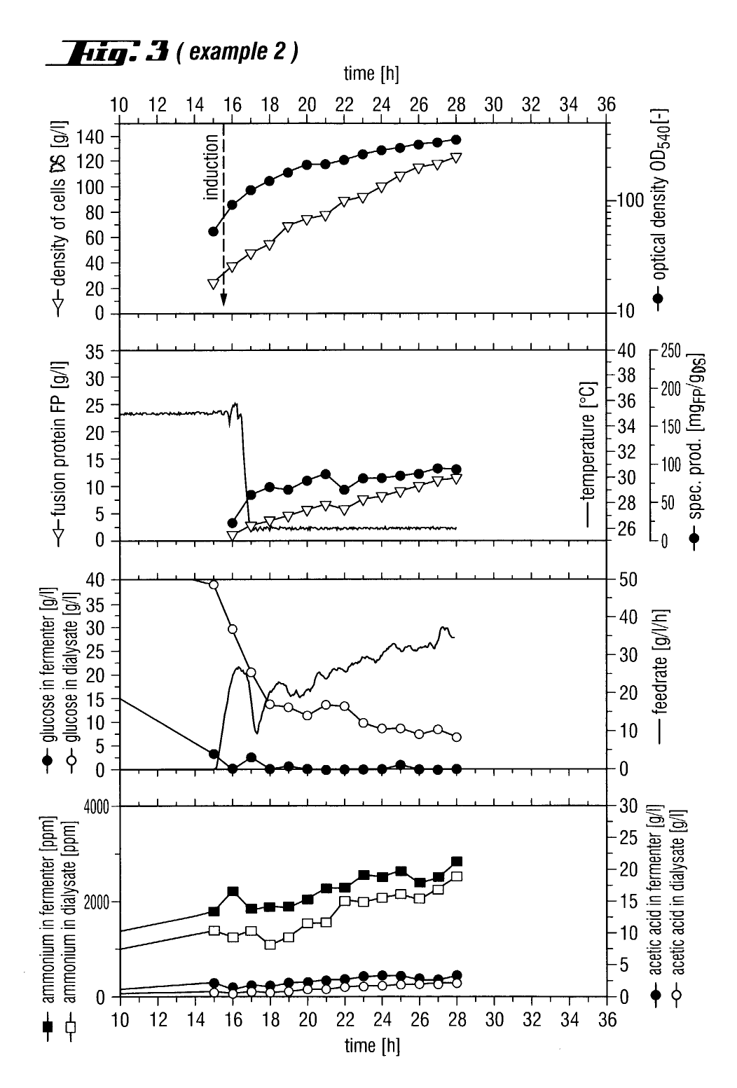

The concentration of the dissolved oxygen pO₂ was adjusted to a value of >10% oxygen saturation by mixing air and O₂ at a total flow of 50 I_{N}/h. The fermentation ended after 28 hours. The maximum dry substance amounted to 122.8 g/l and the product concentration was 11.6 g of fusion protein/l. The chronological course of the fermentation is depicted in FIG. 3. At the time of the induction, the maximum specific growth rate was μₘₐₓ=0.575/h. After the temperature had been lowered down to 26° C., it immediately fell abruptly down to μ=0.298/h. The growth rate subsequently decelerated steadily until the end of the fermentation, down to a value of μ=0.046/h. The acetate concentration was 3.5 g/l.

### Example 3

### Comparison of Examples 1 and 2

While the experimental fermentation cited in Example 2 achieved a relatively high dry biomass, at a value of 122.8 g/l, the content of fusion protein, at a value of 11.6 g/l, was lower than that in Example 1. At a constant fermentation temperature of 35° C., fermentation in Example 1 achieved a dry biomass of 84 g/l and a fusion protein content of 15.8 g/l. A comparison of the specific productivity (mg of fusion protein (FP)/g of dry biomass (DBM)) in the two fermentations shows that this productivity is markedly worse in the case of Example 2. While a specific productivity of 186 mg of FP/g of DBM was generated in Example 1, this productivity was only 95 mg of FP/g of DBM in Example 2. This shows clearly that, while the rapid temperature shift has a positive effect on cell growth and acetate formation, it exerts a negative effect on product formation.

### Experimental Conditions for Example 4

Subsequent experiments investigating the influence of temperature on protein expression in E. coli were carried out in a Bioengineering laboratory dialysis reactor having an outer Naturin film of a thickness of 80 μm. A cuprophane dialysis membrane, having a wall thickness of 20 μm and a molecular cut-off of 10 kDal, was used for separating the fermentation space and the dialysate space. The exchange area of the membrane amounted to 50 m²/m³ of cell suspension. At the beginning of the culture, an initial volume of 1.1 L was introduced into the inner space in the fermenter while 3.6L of dialysate were introduced into the outer space in the fermenter. As an additional dialysis, 2 liters of once-concentrated medium was substituted in the outer space of the fermenter during the course of the fermentation. The organization of the experiment is depicted diagramatically in FIG. 1.

Preliminary Culture and Inoculum

The inoculation of the preliminary culture took place, under a laminar air flow hood, in a 500 ml shaker flask, which was fitted with baffles and filled with 100 ml of the sterilized preliminary culture medium, using a working seed (1 ml) which had been cryostated at −80° C.

Composition based on 1 L of medium is the same as in Examples 1 and 2

The preliminary culture was incubated for 3.25 h, at 37° C. and 200 rpm, on an orbital shaker.

After a 1 ml sample had been removed, the optical density at 540 nm was determined using a 1:10 dilution. The measured opt. density is used to compute the inoculation volume in each case.

Fermentation medium is the same as in Examples 1 and 2

Prior to sterilization, the pH was adjusted to 3.5 using 33% NaOH. After the sterilization, the pH was adjusted to 7.1 using 25% NH₃ solution. After sterilization, 5 ml of thiamine HCl (5 g/l) were added to the medium in the inner space.

Trace element solution (100-fold conc.) is the same as in Examples 1 and 2.

Preparing the Fermenter Unit

The unit was sterilized in situ, together with the fermentation medium, at 121° C. for 20 min. Prior to the sterilization, the volume in the inner space was 1,100 ml while that in the outer space was 3,900 ml. The connections for gassing, for exhaust air, for withdrawing samples from the inner space and the outer space, and for adding concentrated feed and ammonia solution, were sterilized separately in the autoclave at 121° C. for 20 min and, after the fermenter had been sterilized, attached using diaphragms while flaming. After sterilization, medium was drained from the inner space in order to compensate for medium, which had passed from the outer space to the inner space during the sterilization, and to adjust the volume once again to 1,100 ml. The pH was adjusted to 7.1 using 25% NH₃ solution. 5 ml of thiamine HCl solution (5 g/l) was added to the inner space.

### Example 4

### Optimized Temperature Profile

The sterilized medium was inoculated with 7 ml of inoculum using a sterile tip while flaming. The fermentation was operated batchwise until the glucose concentration in the inner space in the fermenter had fallen below a value of 3 g/l. After the experiment had been in progress for 18.1 hours, concentrated glucose solution was sampled into the inner space in the fermenter using a Watson Marlow peristaltic pump. The concentration of glucose in the inner space in the fermenter was subsequently determined irregularly and the substrate concentration was kept in the range of from 0.5 to 1.0 g of glucose/l by manually adjusting the pump speed.

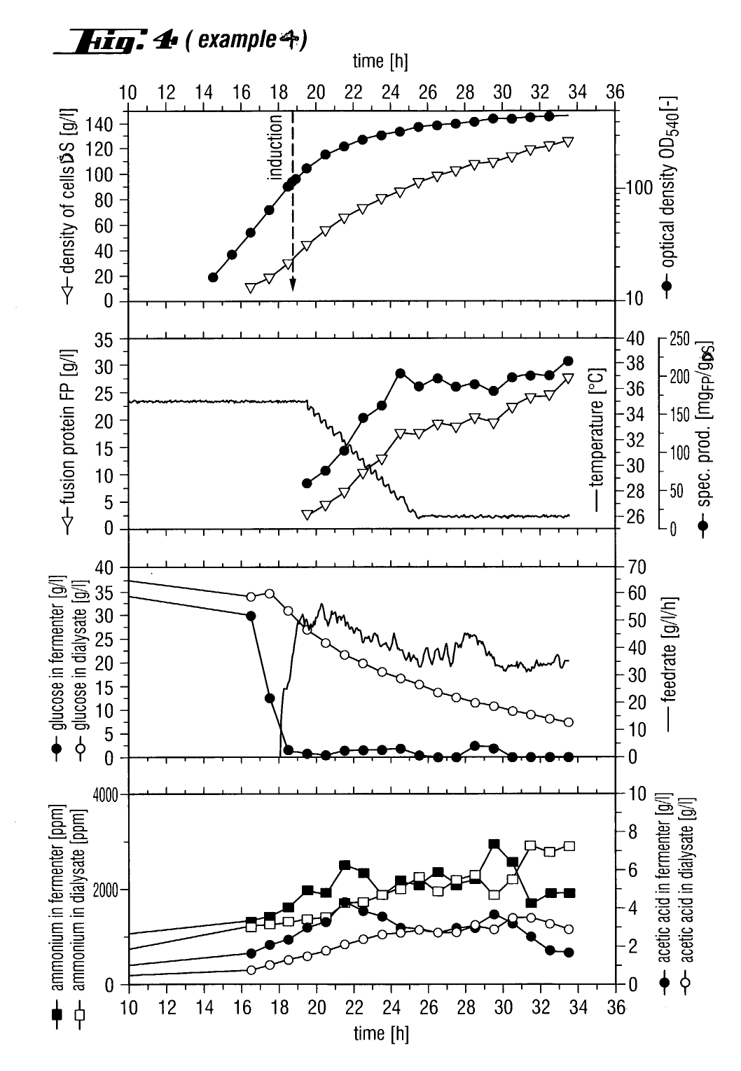

After 18.8 hours, the induction was effected, at an optical density of OD₅₄₀=94, by adding 3.0 ml of 30% IPTG solution, which was sterilized by filtration, to the inner space in the fermenter and adding 10.0 ml of 30% IPTG solution to the outer space in the fermenter. The stirrer speed was 1,500 rpm in the inner space in the fermenter and 800 rpm in the outer space in the fermenter. There was no further variation in the stirrer speed during the course of the fermentation. Up to 40 min after the induction (19.5 hours), the temperature was constant at 35° C. The temperature was then adjusted, in steps of 0.5° C., after in each case 20 min, down to the final value of 26° C. and then maintained constant until the end of the fermentation. The downshifted temperature of 26° C. was reached after 5.7 hours (running time 25.2 hours). The pH was measured using a Broadley combination electrode. The pH was adjusted online to 7.1 with the aid of a Bioengineering pH controller and by metering in 25% NH₃ solution using a Meredes peristaltic pump. The concentration of the dissolved oxygen pO₂ was adjusted to a value of >10% oxygen saturation by mixing air and O₂ at a total flow of 50 I_{N}/h. The fermentation ended at 33.5 hours. The maximum dry substance was 125.9 g/l and the product concentration was 27.9 g of fusion protein/l. The chronological course of the fermentation is depicted in FIG. 4. At the time of the induction, the maximum specific growth rate was μₘₐₓ=0.418/h. The specific growth rate decelerated in line with the fall in temperature. After 1 hour of expression, μ was=0.342/h. After that, there was a continuous deceleration down to μ_{26° C.}=0.119/h. At the end of the fermentation, after an expression phase of 14 hours, μ was=0.016/h. The acetate content was 4 g/l.

### Example 5

### Comparison of the Results of Examples 1, 2 and 4

The experimental fermentations clearly verified the influence of shifting the temperature from 35° C. down to 26° C. on the growth behavior of the microorganisms and on productivity. A slow and steady temperature shift, which was adapted to the specific growth rate of the microorganisms had a positive effect both on growth and on product formation. It was also found that, when the temperature shift is rapid, the specific growth rate decreases rapidly, by approximately half (μ_{35° C.}=0.575/h down to μ_{26° C.}=0.298/h) immediately after the temperature has been lowered. By contrast, when the temperature shift is slow, the specific growth rate decreases continuously, like it does in fermentations without any temperature shifting. The optimized, slow temperature shifting not only had a positive effect on cell growth; but also markedly improved product formation and quality. In the experimental fermentation, 125.9 g of DBM/l and 27.9 g of FP/l were produced and the specific product quality improved from a value of approximately 180 mg of FP/g of DBM, as seen in normal fed-batch fermentations, to a value of 221.6 mg of FP/g of DBM. When the temperature was shifted rapidly, as is customary in the state of the art, the specific product quality deteriorated down to 95 mg of FP/g of DBM.

Fermentations, which are conducted at low temperatures, produce fewer by-products. In the experimental fermentations, this was clearly demonstrated by the acetate formation. The experimental fermenter which was fermented at 35° C. (Example 1), formed 16.5 g of acetate/l whereas, by contrast, the fermentations which were conducted at 26° C. only produced 3.5 g/l (Example 2) and 4 g/l (Example 4).

The instant invention, in which the slow, step-wise lowering of the temperature was aligned with the growth behavior of the microorganism did not lead to the bioprocess coming to a standstill. Instead, the lowering of the temperature down to 26° C. decelerated cell growth and metabolism. Unexpectedly, this had a positive effect on product formation and on acetate formation. It was possible to increase the product concentration to 27.9 g of FP/l while achieving a markedly superior specific protein productivity. Comparison with industrial fed-batch fermentations (7 g/l) shows a four-fold increase in yields. Accordingly, superior fermentation conditions have been achieved in the production of heterologous protein expressed in E. coli cells by practicing the invention described herein.

### Other Embodiments

It is to be understood that while the invention has been described in conjunction with the detailed description thereof, that the foregoing description is intended to illustrate and not limit the scope of the invention, which is defined by the scope of the appended claims. Other aspects, advantages, and modifications are within the scope of the following claims. All references cited herein are incorporated herein by reference.

We claim:

1. A fermentation method for improving the expression of a heterologous protein in E. coli cells comprising the steps of:
i. growing E. coli cells in culture in the growth phase until the cell mass is predominantly at maximum specific growth rate, and
ii. slowly shifting the temperature downward in the expression phase from 35° C. down to 26° C. starting from 20 to 60 minutes after induction in 0.2° C. to 0.8° C. temperature increments at 10 to 30 minute intervals such that said expression of heterologous protein is increased.

2. The method of claim 1 wherein the temperature is shifted downward beginning 40 minutes after induction.

3. The method of claim 1 wherein said intervals are 20 minutes.

4. The method of claim 1 wherein said temperature increment is 0.5° C.

5. The method of claim 1 wherein the E. coli cells are-cultured in membrane dialysis fermenters which have a capacity of more than 2 liters.

6. The method of claim 5 wherein said dialysis membrane is installed internally or externally on the fermenter

7. The method of claim 6 wherein said membranes have a cut-off of at least 10 kDal.

8. The method of claim 1 wherein the induction is effected at an optical density of from 90 to 110.

9. The method of claim 8 wherein said optical density is 100.

10. The method of claim 1 wherein after the temperature has been shifted down to 26° C. the temperature is maintained until the end of the culture.

11. The method of claim 1 wherein a minimal medium is used.

12. The method of claim 1 wherein the carbohydrate concentration is from 0.1 to 1.0 g/l.

13. The method of claim 1 wherein the oxygen saturation is >10%.
